(19) United States
(12) Patent Application Publication
(10) Pub. No.: US 2011/0052522 A1
(43) Pub. Date: Mar. 3, 2011

(54) COSMETICS COMPRISING CISTUS PLANT EXTRACTS

(75) Inventors: Yvonne Schiemann, Essen (DE); Mike Farwick, Essen (DE); Heike Blasko-Begoihn, Essen (DE); Peter Lersch, Dinslaken (DE)

(73) Assignee: EVONIK GOLDSCHMIDT GMBH, Essen (DE)

(21) Appl. No.: 12/990,970

(22) PCT Filed: Apr. 6, 2009
(86) PCT No.: PCT/EP09/54068

§ 371 Date: Nov. 4, 2010

(30) Foreign Application Priority Data
May 6, 2008 (DE) 102008022392.1

(51) Int. Cl. A61K 36/185; A61K 8/97; A61Q 5/00; A61Q 19/00; A61P 29/00; A61P 17/00

(52) U.S. Cl. 424/74; 424/725

(57) ABSTRACT

The present invention relates to the use of an extract from cistus, prepared by a novel process, as care active ingredient for the care of human or animal body parts, to care formulations and to the use of the care formulations.

### FIELD OF THE INVENTION

The present invention relates to the use of an enriched extract from Herba Cistus ssp. L., prepared by a novel process, as care active ingredient for the care of human or animal body parts, to care formulations and to the use of the care formulations.

### PRIOR ART

In the Cistaceae family, the cistuses form a genus with 18 to 20 species. The subgenus Cistus incanus L. is described as an aromatic evergreen shrub up to 1 m in size. Its alternate egg-shaped-lanceolate leaves and radially symmetrical, five-sepaled flowers are customary for the entire family, although the pink-red, often pink-coloured to pale violet coloration of the flowers is characteristic of this subgenus. The preferred habitat is the eastern Mediterranean region.

Folk medicine used the effect of this plant in very diverse ways, but primarily as adjuvant for topical, often allergy-induced itching, and also for prevention and wound treatment in the case of bacterial infections and mycoses. Traditionally, decoctions of spring plant parts above the ground have been used for this purpose. Earlier publications have reported on the successful use of cistus solutions for the treatment of neurodermitis, acne vulgaris, and also in the case of inflammatory disorders of the mouth and throat cavity.

A more detailed specification into the varieties Cistus incanus L. ssp. creticus and Cistus incanus L. ssp. tauricus has only recently become essential and relevant as a result of a distinction in terms of Plant Breeders' Rights of the variety Cistus incanus L. ssp. Pandalis® or under the trademark Cystus®, which, especially for lay persons, cannot be differentiated from Cistus incanus L: ssp. tauricus.

The historically oldest records for cistus preparations deal with labdanum, a resin which was obtained predominantly from the leaves and branches of Cistus ladaniferus L., Cistus monspliensis L. and Cistus incanus L. On account of its pleasant aroma, it was more likely to be smoked, but also had a medical use as expectorant in cases of respiratory tract catarrh, and also in plasters and unguents for wound treatment.

Phytochemical investigations of the genus Cistus consequently often concentrated on the analysis of the lipophilic constituents, in particular of the essential oil and of the resin constituents. These contain usual structures of terpene metabolism such as mono-, sesqui- and diterpenes, and also alcohols and esters. Further investigations dealt with the polyphenol fractions of the flavonoids and tannins. In this connection, kaempferol, quercetin and apigenin were analysed as the predominant base structures. Our own analyses have revealed that the known pattern is found again in the polyphenol fingerprint: similarly to the green tea extract, epicatechin (EC) and epigallocatechin gallate (EGCG) could be detected in the region of the catechin derivatives. However, the oligomer fraction in the case of this cistus is significantly larger. The total content of polyphenols in the plant is generally given as 4%, although the fraction in the leaves is significantly higher.

It is the aim of care cosmetics to attain the impression of an external youthful appearance, for example that of the skin and hair. In principle, there are various ways to achieve this object. For example, existing damage to the skin, such as irregular pigmentation or wrinkling, can be compensated for by concealing powders or creams. Another approach is to protect the skin against environmental influences which lead to permanent damage and thus ageing of the skin. The idea is thus to intervene in a prophylactic manner and thereby to delay the ageing process. One example thereof are UV filters which, through absorption of certain wavelength regions, avoid or at least reduce skin damage. Whereas in the case of UV filters, the harmful event, the UV radiation, is screened by the skin, another route attempts to support the natural defence and/or repair mechanisms of the skin against the harmful event. Finally, a further approach which is pursued is to compensate for the weakening defence functions of the skin against harmful influences, which occurs with increasing age, by supplying substances externally which are able to replace this diminishing defence and/or repair function. For example, the skin has the ability to capture free radicals which are produced by external or internal stress factors. This ability decreases with increasing age, as a result of which the ageing process accelerates with increasing age. Products for the care of slackened, in particular aged, skin are known per se. They comprise, for example, retinoids (vitamin A acid and/or derivatives thereof) or vitamin A and/or derivatives thereof. Their effect on the structural damage, however, is limited in its extent. Moreover, during product development, there are considerable difficulties associated with stabilizing the active ingredients to an adequate degree against oxidative decomposition. Moreover, the use of vitamin A acid-containing products often causes severe erythematous skin irritations. Retinoids can therefore only be used in low concentrations. Slackened skin is also often linked with an accompanying symptom of obesity and/or the so-called cellulite often associated therewith. The body image of consumers has risen considerably in past years. In this connection, besides cleaning and care applications, measures are also increasingly being taken in order to improve the body silhouette. Cellulite—a widespread phenomenon—assumes a central position here. The visible appearance of cellulite is based on an increase of fatty bodies in the subcutis (subcutaneous fatty tissue), a connective tissue weakening, and a reduction in the perfusion ratios in the blood and lymph pathways. The cause is thus an in part endogenous weakening of the connective tissue with a simultaneous occurrence of enlarged fat cell chambers as a result of excess weight, unbalanced diet, lack of movement. The formation of cellulite can also be attributed to an increased permeability of the capillary walls, which permits the penetration of water into the connective tissue.

The skin is exposed to numerous stresses. Regular contact with potentially skin-irritative or allergising working materials can lead, especially on the hands, to damage of the skin barrier and consequently to the appearance of eczema. When using protective gloves, it must be ensured that the glove material has been adequately tested for resistance to the harmful substances present in each case. Permeable gloves not only offer no protection, but can sometimes even intensify the effect of the harmful material through the occlusion effect of the glove material. Furthermore, protracted wearing of close-fitting gloves can lead to a buildup of heat and moisture and consequently to swelling and softening of the skin with subsequent decomposition of perspiration and odour development. It is clear from this why the prophylaxis must be used during numerous occupational activities to the use of suitable skin protection and skin care preparations and also to the realization of gentle skin cleaning.

Human hair in turn is subjected daily to a very wide variety of influences. Besides mechanical stresses through brushing, combing, putting up or tying back, the hair is also attacked by environmental influences such as, for example, intense UV radiation, cold, wind and water. In particular, however, frequent washing with aggressive surfactants also contributes to greater or lesser damage to the hair structure being caused.

The hair becomes brittle, dry, dull, porous and difficult to comb as a result of treatments with hair colourants or hair tints, frequent washing or UV exposure. It loses moisture, elasticity and in particular mechanical resistance and tear resistance. This is evident from a significant decrease in the stress-strain forces and the tear forces on wet hair. Moreover, it is less resistant to further damage by chemicals, surfactants and environmental influences than healthy hair.

There are special preparations for the treatment of such damaged hair, such as, for example, hair rinses, hair treatments, shampoos, leave-in conditioners etc., which, however, can in particular improve the combability, the feel and the shine of damaged hair. Several plant extracts such as, for example, extracts from seaweed, stinging nettles, eucalyptus, rosemary and wheatgerm have become known for their hair care properties. A further known way of treating the described problems, especially those of the skin, consists in adding antioxidants to the cosmetic preparations. The effect of the antioxidants is that they act as free-radical scavengers for the free radicals which are produced during autooxidation.

The antioxidative capacity of a sample can be quoted using the Trolox Equivalent Antioxidative Capacity (TEAC). During measurement, 6-hydroxy-2,5,7,8-tetramethylchroman-2-carboxylic acid (Trolox) serves as reference, for which reason the result is quoted in Trolox equivalents. The principle of the measurement is based on the fact that an oxidative medium is produced in a reaction vessel and an added antioxidant slows the oxidation reaction. Antioxidants may be, for example, the polyphenols present in the sample to be measured. The course of the oxidation reaction over time is measured and compared with that of Trolox. The course of the oxidation reaction is measured inter alia using a photometer and the chemical auxiliary 2,2′-azinobis(3-ethylbenzothiazoline-6-sulphonic acid) (ABTS), which, in oxidative medium forms a stable green-coloured free-radical cation which can be measured photometrically at 734 nm.

Oxygen Radical Absorbance Capacity (ORAC) is an alternative measurement method which can be used which was developed by American researchers for characterizing antioxidative properties of foods. Using this method, a differentiation into hydrophilic and lipophilic fractions of the antioxidative effect is possible. For better comparability, the unit of the Trolox equivalent (TE) was retained here.

There is furthermore a growing need for new, further and improved active ingredients for cosmetic hair and skin treatment and also for hair after-treatment which, alone or in combination with the respective cleaning and/or care agents, optionally with co-use of customary care and physiologically favourable active ingredients and additives such as, for example, vitamins, ceramides, sphingosides, lecithins, antioxidants, UV filters and others, have a broad care and conditioning effect.

It was the object of the invention to provide a care active ingredient which has a high antioxidative capacity, can be readily formulated and has further properties required for the care of human or animal body parts, with hair being considered a body part.

### DESCRIPTION OF THE INVENTION

Surprisingly, it has been found that the extracts from Herba Cistus ssp. L. described below achieve the object mentioned above.

The present invention therefore provides the use of an extract from Herba Cistus ssp. L., prepared by a process as described in claim 1, as care active ingredient for the care of human or animal body parts, to care formulations and to the use of the care formulations.

One advantage of the invention is the unexpectedly high antioxidative capacity of the care formulations according to the invention.

A further advantage is the colouration of the care active ingredient itself, which is not troublesome in the care formulations.

Another advantage of the invention is the good availability of the raw material, i.e. of the plant, on account of the plant being easy to cultivate.

The invention provides the use of an extract from cistus, prepared by the process steps
extraction from Herba Cistus ssp. L. with an extractant selected from the group water, alcohols and mixtures thereof
removal of extraction residues
at least partial removal of the extractant
redissolution in an aqueous solvent and removal of insoluble constituents
selective enrichment through a solid-phase extraction
   as care active ingredient for the care of human or animal body parts.

A virtually constant quality of the plant feed material is desirable for such a production process. This is achieved through analysis of the polyphenol contents (determined using Folin's reagent and calculated as gallic acid analogously to the methodology in the European Pharmacopoeia, chapter PH. EUR. (2.8.14)) and mixing of batches.

Preferably, the drug is subjected to grinding and wind sifting in order to achieve an enrichment of the leaf material as opposed to the stalk fractions. The leaves have higher contents of polyphenols. As a result of this, an improvement by ca. 30% is achieved. Plant parts of Cistus incanus L. ssp tauricus above the earth are preferably used.

A content of min. 12% polyphenols is preferred as starting material.

Suitable extractants are in particular water, methanol, ethanol, 1-propanol, 2-propanol and mixtures thereof. A water-containing extractant is preferred. The content of alcohol is preferably not more than 50% (v/v).

In one embodiment, the extraction is carried out at an elevated temperature. Temperatures of from 40 to 80° C. are particularly preferred in order to achieve a particularly high polyphenol yield.

After the extraction, the drug residue is usually removed; this can be carried out, for example, by filtering it off or removing it with suction and then squeezing out the drug residue. Further options are known to the person skilled in the art.

The extractant is then at least partially removed from the resulting extract. This can be carried out, for example, by drawing off the solvent in a rotary evaporator or with the aid of a plate evaporator. A gentle treatment is preferred.

Following removal, the dry substance fraction of the remaining extract is preferably more than 50% (m/m).

In the next step, the remaining residue is redissolved. For the redissolution, especially water or mixtures of water with alcohol are particularly suitable. Preferably, the redissolution agent comprises at least 50% (m/m) water.

After the redissolution, a residue remains which is separated off by filtration, drawing off with suction, decantation or the like and can be discarded. The residue comprises at least one part of the tannins (less bioactive polyphenols).

An enrichment step then takes place through a solid-phase extraction. An extraction with adsorber resins is particularly suitable. Typical adsorber resins are, for example, non-ionic hydrophobic divinylbenzene copolymers, aliphatic ester polymers and formophenol polymers. Such adsorbers are commercially available under the tradename Amberlite®. Suitable products are the grades XAD2, XAD4, XAD7HP, XAD16, XAD761 or XAD1180. It is, however, also possible to use analogously characterized resins from other manufacturers, such as from Diaion (SP series), from Bayer (Lewatite®) or from Miontech (P series).

The resin grade Amberlite® XAD7HP is particularly preferred here.

The cistus plant is preferably Cistus incanus, particularly preferably Cistus incanus L. ssp. tauricus.

The care active ingredient according to the invention, obtainable by the process steps
extraction of plant parts from Cistus L. with an extractant selected from the group water, alcohols and mixtures thereof
removal of extraction residues
at least partial removal of the extractant
redissolution in an aqueous solvent and removal of insoluble constituents
selective enrichment through a solid-phase extraction, has a content of polyphenols of more than 30 catechin equivalent percent, preferably more than 50 catechin equivalent percent, particularly preferably more than 60 catechin equivalent percent polyphenols based on the total mass of the care active ingredient.

To determine the content of polyphenols (in catechin equivalent percent), a quantitative photometric determination based on Folin-Ciocalteurs phenol reagent with (+−)-catechin hydrate as reference substance is used:

### Reference Solutions

30 mg of catechin hydrate are dissolved in 10% strength ethanol and then diluted with the same solvent to 50 ml (stock solution).

7.0 ml or 4.0 ml (depending on the polyphenol concentration of the substance to be investigated) of this stock solution are diluted to 10 ml with 10% strength ethanol this solution serves as reference solution for establishing a calibration curve.

The extract to be determined is dissolved in ethanol (96%) and topped up to a defined volume (e.g. 250 ml) with water. 250 μl of this sample or 250 μl of reference substance ((+−)-catechin hydrate (Sigma, Art. C 1788, Ch. 045K1052) in 10% ethanol) are placed in a 25 ml measuring flask and diluted with 5 ml of water. 1 ml of Folin-Ciocalteurs phenol reagent (Merck Art. No. 1.09001) and 2.5 ml of 30% strength sodium carbonate solution are then added and the measuring flask is topped up to 25 ml with water. After 60 min, the absorption of the solutions is measured at 720 nm against water as compensation liquid.

The total polyphenol content is determined with the help of the calibration function of the calibration curves. Y=AX+B

X=(Y−B)/A

Total polyphenols as (+−)-catechin hydrate (%)=X/C
Y=Absorption of the measurement solution at 720 nm
C=Concentration of the measurement solution (mg/ml)

The total polyphenols can thus be quoted as % of (+−)-catechin hydrate “Catechin Equivalent Per cent”.

The invention further provides topical care formulations comprising the care active ingredient according to the invention.

The care formulations according to the invention are preferably characterized in that these constitute cosmetic, dermatological or pharmaceutical formulations.

The care formulations according to the invention comprise from 0.01 mass percent to 20 mass percent, preferably 0.05 mass percent to 5 mass percent, particularly preferably 0.1 mass percent to 2 mass percent of care active ingredient, based on the total mass of the care formulation.

The care formulations according to the invention can comprise, for example, at least one additional component selected from the group of

### Emollients,

Emulsifiers and surfactants,
Thickeners/viscosity regulators/stabilizers,
UV photoprotective filters,
Antioxidants and vitamins,
Hydrotropes (or polyols),
Solids and fillers,
Film formers,
Pearlescent additives,
Deodorant and antiperspirant active ingredients,
Insect repellents,
Self-tanning agents,

### Preservatives,

### Conditioners,

### Perfumes,

### Dyes,

Biogenic active ingredients,
Care additives,
Superfatting agents,

### Solvents.

Emollients which can be used are all cosmetic oils, in particular mono- or diesters of linear and/or branched mono- and/or dicarboxylic acids having 2 to 44 carbon atoms with linear and/or branched saturated or unsaturated alcohols having 1 to 22 carbon atoms. It is likewise possible to use the esterification products of aliphatic, difunctional alcohols having 2 to 36 carbon atoms with monofunctional aliphatic carboxylic acids having 1 to 22 carbon atoms. Also suitable are long-chain aryl acid esters, such as, for example, esters of benzoic acid, e.g. benzoic acid esters of linear or branched, saturated or unsaturated alcohols having 1 to 22 carbon atoms, or else isostearyl benzoate or octyldodecyl benzoate. Further monoesters suitable as emollients and oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g. esters of unsaturated fatty alcohols, having 12 to 22 carbon atoms and saturated and unsaturated fatty acids having 12 to 22 carbon atoms, as are accessible from animal and vegetable fats. Also suitable, however, are naturally occurring monoester and/or wax ester mixtures, as are present, for example in jojoba oil or in sperm oil. Suitable dicarboxylic acid esters are, for example, di-n-butyl adipate, di-n-butyl sebacate, di(2-ethylhexyl)-adipate, di(2-hexyldecyl) succinate, diisotridecyl azelate. Suitable diol esters are, for example, ethylene glycol dioleate, ethylene glycol diisotridecanoate, propylene glycol di(2-ethylhexanoate), butanediol diisostearate, butanediol dicaprylate/caprate and neopentyl glycol dicaprylate. Further fatty acid esters which can be used as emollients are, for example, C₁₂-C₁₅ alkyl benzoate, dicaprylyl carbonate, diethylhexyl carbonate. Emollients and oil components which can likewise be used are longer-chain triglycerides, i.e. triple esters of glycerol with three acid molecules, of which at least one is relatively long-chain. By way of example, mention may be made here of fatty acid triglycerides; examples of such which may be used are natural, vegetable oils, e.g. olive oil, sunflower oil, soybean oil, peanut oil, rapeseed oil, almond oil, sesame oil, avocado oil, castor oil, cocoa butter, palm oil, but also the liquid fractions of coconut oil or of palm kernel oil, and also animal oils, such as, for example, shark liver oil, cod liver oil, whale oil, beef tallow and butter fat, waxes such as beeswax, carnauba palm wax, spermaceti, lanolin and claw oil, the liquid fractions of beef tallow and also synthetic triglycerides of capryl/capric acid mixtures, triglycerides of technical-grade oleic acid, triglycerides with isostearic acid, or from palmitic acid/oleic acid mixtures as emollients and oil components. Furthermore, hydrocarbons, in particular also liquid paraffins and isoparaffins, can be used. Examples of hydrocarbons which can be used are paraffin oil, isohexadecane, polydecene, vaseline, Paraffinum perliquidum, squalane, ceresine. Furthermore, it is also possible to use linear or branched fatty alcohols such as oleyl alcohol or octyldodecanol, and also fatty alcohol ethers such as dicaprylyl ether. Suitable silicone oils and silicone waxes are, for example, polydimethylsiloxanes, cyclomethylsiloxanes, and also aryl- or alkyl- or alkoxy-substituted polymethylsiloxanes or cyclomethylsiloxanes. Suitable further oil bodies are, for example, Guerbet alcohols based on fatty alcohols having 6 to 18, preferably 8 to 10, carbon atoms, esters of linear C6-C22-fatty acids with linear C6-C22-fatty alcohols, esters of branched C6-C13-carboxylic acids with linear C6-C22-fatty alcohols, esters of linear C6-C22-fatty acids with branched C8-C18-alcohols, in particular 2-ethylhexanol or isononanol, esters of branched C6-C13-carboxylic acids with branched alcohols, in particular 2-ethylhexanol or isononanol, esters of linear and/or branched fatty acids with polyhydric alcohols (such as, for example, propylene glycol, dimerdiol or trimertriol) and/or Guerbet alcohols, triglycerides based on C6-C10-fatty acids, liquid mono-/di-/triglyceride mixtures based on C6-C18-fatty acids, esters of C6-C22-fatty alcohols and/or Guerbet alcohols with aromatic carboxylic acids, in particular benzoic acid, vegetable oils, branched primary alcohols, substituted cyclohexanes, linear C6-C22-fatty alcohol carbonates, Guerbet carbonates, esters of benzoic acid with linear and/or branched C6-C22-alcohols (e.g. Finsolv™ TN), dialkyl ethers, ring-opening products of epoxidized fatty acid esters with polyols, silicone oils and/or aliphatic or naphthenic hydrocarbons.

Emulsifiers or surfactants which may be used are non-ionic, anionic, cationic or amphoteric surfactants.

Nonionogenic emulsifiers or surfactants which can be used are compounds from at least one of the following groups:

addition products of from 2 to 100 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,

C_{12/18}-fatty acid mono- and diesters of addition products of from 1 to 100 mol of ethylene oxide onto glycerol,

glycerol mono- and diesters and sorbitan mono- and diesters of saturated and unsaturated fatty acids having 6 to 22 carbon atoms and ethylene oxide addition products thereof,

alkyl mono- and oligoglycosides having 8 to 22 carbon atoms in the alkyl radical and ethylene oxide addition products thereof,

addition products of from 2 to 200 mol of ethylene oxide onto castor oil and/or hydrogenated castor oil,

partial esters based on linear, branched, unsaturated or saturated C₆-C₂₂-fatty acids, ricinoleic acid, and 12-hydroxystearic acid and glycerol, polyglycerol, pentaerythritol, dipentaerythritol, sugar alcohols (e.g. sorbitol),

alkyl glucosides (e.g. methyl glucoside, butyl glucoside, lauryl glucoside) and polyglucosides (e.g. cellulose),

mono-, di- and trialkylphosphates, and mono-, di- and/or tri-PEG alkyl phosphates and salts thereof,

polysiloxane-polyether copolymers (dimethicone copolyols), such as, for example PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15, polysiloxane-polyalkyl-polyether copolymers and corresponding derivatives, such as, for example, lauryl or cetyl dimethicone copolyols, in particular cetyl PEG/PPG-10/1 dimethicone (ABIL® EM 90 (Evonik)),

mixed esters of pentaerythritol, fatty acids, citric acid and fatty alcohol as in DE 11 65 574 and/or mixed esters of fatty acids having 6 to 22 carbon atoms, methylglucose and polyols, such as, for example, glycerol or polyglycerol,

citric acid esters, such as, for example, glyceryl stearate citrate, glyceryl oleate citrate and dilauryl citrate.

Anionic emulsifiers or surfactants can contain water-solubilizing anionic groups, such as, for example, a carboxylate, sulphate, sulphonate or phosphate group and a lipophilic radical. Skin-compatible anionic surfactants are known to the person skilled in the art in large numbers and are commercially available. Here, these may be alkyl sulphates or alkyl phosphates in the form of their alkali metal, ammonium or alkanolammonium salts, alkyl ether sulphates, alkyl ether carboxylates, acyl sarcosinates, and sulphosuccinates and acyl glutamates in the form of their alkali metal or ammonium salts.

Cationic emulsifiers and surfactants can also be added. Those which can be used are, in particular, quaternary ammonium compounds, in particular those provided with at least one linear and/or branched, saturated or unsaturated alkyl chain having 8 to 22 carbon atoms, such as, for example, alkyltrimethylammonium halides, such as, for example, cetyltrimethylammonium chloride or bromide or behenyltrimethylammonium chloride, but also dialkyldimethylammonium halides, such as, for example, distearyldimethylammonium chloride. Furthermore, monoalkylamidoquats such as, for example, palmitamidopropyltrimethylammonium chloride or corresponding dialkylamidoquats, can be used.

Furthermore, readily biodegradable quaternary ester compounds can be used; these may be quaternized fatty acid esters based on mono-, di- or triethanolamine. Furthermore, alkylguanidinium salts can be added as cationic emulsifiers.

Typical examples of mild, i.e. particularly skin-compatible, surfactants are fatty alcohol polyglycol ether sulphates, monoglyceride sulphates, mono- and/or dialkyl sulphosuccinates, fatty acid isethionates, fatty acid sarcosinates, fatty acid taurides, fatty acid glutamates, ether carboxylic acids, alkyl oligoglucosides, fatty acid glucamides, alkylamidobetaines and/or protein fatty acid condensates, the latter for example based on wheat proteins.

Furthermore, it is possible to use amphoteric surfactants, such as, for example, betaines, amphoacetates or amphopropionates, thus, for example, substances such as the N-alkyl-N,N-dimethylammonium glycinates, for example cocoalkyldimethylammonium glycinate, N-acylaminopropyl-N,N-dimethylammonium glycinates, for example cocoacylaminopropyl-dimethylammonium glycinate, and 2-alkyl-3-carboxymethyl-3-hydroxyethylimidazolines having in each case 8 to 18 carbon atoms in the alkyl or acyl group, and also cocoacylaminoethyl hydroxyethylcarboxymethyl glycinate.

Of the ampholytic surfactants, it is possible to use those surface-active compounds which, apart from a C8/18-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylaminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Further examples of ampholytic surfactants are N-cocoalkylaminopropionate, cocoacylaminoethylaminopropionate and C12/18-acylsarcosine.

Suitable thickeners are, for example, polysaccharides, in particular xanthan gum, guar-guar, agar agar, alginates and tyloses, carboxymethylcellulose and hydroxyethylcellulose, also relatively high molecular weight polyethylene glycol mono- and diesters of fatty acids, polyacrylates (e.g. Carbopols™ or Synthalens™), polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homologue distribution or alkyl oligoglucosides, and also electrolytes such as sodium chloride and ammonium chloride.

Suitable thickeners for thickening oil phases are all thickeners known to the person skilled in the art. In particular, mention is to be made here of waxes, such as hydrogenated castor wax, beeswax or microwax. Furthermore, inorganic thickeners can also be used, such as silica, alumina or sheet silicates (e.g. hectorite, laponite, saponite). In this connection, these inorganic oil phase thickeners may be hydrophobically modified. For the thickening/stabilization of water-in-oil emulsions, in particular aerosils, sheet silicates and/or metal salts of fatty acids, such as, for example, zinc stearate, can be used here.

Viscosity regulators for aqueous surfactant systems which may be present are, for example NaCl, low molecular weight non-ionic surfactants, such as cocoamide DEA/MEA and laureth-3, or polymeric, high molecular weight, associative, highly ethoxylate fat derivatives, such as PEG-200 hydrogenated glyceryl palmate.

UV photoprotective filters which can be used are, for example, organic substances which are able to absorb ultraviolet rays and which give off the absorbed energy again in the form of longer-wave radiation, e.g. heat. UVB filters may be oil-soluble or water-soluble. Examples of oil-soluble UVB photoprotective filters are:
3-benzylidenecamphor and derivatives thereof, e.g. 3-(4-methylbenzylidene)camphor,
4-aminobenzoic acid derivatives, such as, for example, 2-ethylhexyl 4-(dimethylamino)benzoate, 2-ethylhexyl 4-(dimethylamino)benzoate and amyl 4-(dimethylamino)benzoate,
esters of cinnamic acid, such as 2-ethylhexyl 4-methoxycinnamate, isopentyl 4-methoxycinnamate, 2-ethylhexyl 2-cyano-3-phenylcinnamate (octocrylene),
esters of salicylic acid, such as, for example, 2-ethylhexyl salicylate, 4-isopropylbenzyl salicylate, homomethyl salicylate,
derivatives of benzophenone, such as, for example, 2-hydroxy-4-methoxybenzophenone,
2-hydroxy-4-methoxy-4′-methylbenzophenone, 2,2′-dihydroxy-4-methoxybenzophenone,
esters of benzalmalonic acid, such as, for example, di-2-ethylhexyl 4-methoxybenzmalonate,
triazine derivatives, such as, for example, 2,4,6-trianilino-(p-carbo-2′-ethyl-1′-hexyloxy)-1,3,5-triazine and octyltriazone,
propane-1,3-diones, such as, for example, 1-(4-tert-butylphenyl)-3-(4′-methoxyphenyl)propane-1,3-dione.

Suitable water-soluble UVB photoprotective filters are:
2-phenylbenzimidazole-5-sulphonic acid and the alkali metal, alkaline earth metal, ammonium, alkylammonium, alkanolammonium and glucammonium salts thereof,
sulphonic acid derivatives of benzophenone, such as, for example, 2-hydroxy-4-methoxybenzophenone-5-sulphonic acid and its salts, sulphonic acid derivatives of 3-benzylidenecamphor, such as, for example, 4-(2-oxo-3-bornylidenemethyl)benzenesulphonic acid and 2-methyl-5-(2-oxo-3-bornylidene)sulphonic acid and salts thereof.

Suitable typical UVA photoprotective filters are in particular derivatives of benzoylmethane, such as, for example, 1-(4′-tert-butylphenyl)-3-(4′-methoxyphenyl)propane-1,3-dione or 1-phenyl-3-(4′-isopropylphenyl)propane-1,3-dione. The UV-A and UV-B filters can of course also be used in mixtures.

Besides the specified soluble substances, insoluble pigments, namely finely disperse metal oxides or salts are also suitable for this purpose, such as, for example, titanium dioxide, zinc oxide, iron oxide, aluminium oxide, cerium oxide, zirconium oxide, silicates (talc), barium sulphate and zinc stearate. The particles here should have an average diameter of less than 100 nm, e.g. between 5 and 50 nm and in particular between 15 and 30 nm. They can have a spherical shape, although it is also possible to use those particles which have an ellipsoidal shape or a shape which deviates in some other way from the spherical form. A relatively new class of photoprotective filters are micronized organic pigments, such as, for example, 2,2′-methylenebis{6-(2H-benzotriazol-2-yl)-4-(1,1,3,3-tetramethylbutyl)phenol} with a particle size of <200 nm, which is obtainable, for example, as 50% strength aqueous dispersion.

Further suitable UV photoprotective filters can be found in the overview by P. Finkel in SÖFW-Journal 122, 543 (1996).

Besides the two aforementioned groups of primary UV photoprotective filters, it is also possible to use secondary photoprotective agents of the antioxidant type which interrupt the photochemical reaction chain which is triggered when UV radiation penetrates into the skin.

Antioxidants and vitamins which can be used are, for example, superoxide-dismutase, tocopherol (vitamin E), tocopherol sorbate, tocopherol acetate, other esters of tocopherol, dibutylhydroxytoluene and ascorbic acid (vitamin C) and its salts, and also derivatives thereof (e.g. magnesium ascorbyl phosphate, sodium ascorbyl phosphate, ascorbyl sorbate), ascorbyl esters of fatty acids, butylated hydroxybenzoic acid and its salts, peroxides, such as, for example, hydrogen peroxide, perborates, thioglycolates, persulphate salts, 6-hydroxy-2,5,7,8-tetramethylchroman-2-carboxylic acid (TROLOX®), gallic acid and its alkyl esters, uric acid and its salts and alkyl esters, sorbic acid and its salts, lipoic acid, ferulic acid, amines (e.g. N,N-diethylhydroxylamine, aminoguanidines), sulphhydryl compounds (e.g. glutathione), dihydroxy-fumaric acid and its salts, glycine pidolate, arginine pilolate, nordihydroguaiaretic acid, bioflavonoids, curcumin, lysine, L-methionine, proline, superoxide dismutase, silymarin, tea extract, grapefruit peel/pip extract, melanin, rosemary extract, thiooctanoic acid, resveratrol, oxyresveratrol, etc.

Hydrotropes which can be used for improving the flow behaviour and the application properties are, for example, ethanol, isopropyl alcohol or polyols. Polyols which are suitable here can have 2 to 15 carbon atoms and at least 2 hydroxyl groups.

### Typical Examples are:

glycerol alkylene glycols, such as, for example, ethylene glycol, diethylene glycol, propylene glycol, butylene glycol, hexylene glycol, and polyethylene glycols with an average molecular weight of from 100 to 1,000 daltons, technical-grade oligoglycerol mixtures with a degree of self-condensation of from 1.5 to 10, such as, for example, technical-grade diglycerol mixtures with a diglycerol content of from 40 to 50% by weight, methylol compounds, such as in particular trimethylolethane, trimethylolpropane, trimethylolbutane, pentaerythritol and dipentaerythritol, lower alkyl glucosides, in particular those with 1 to 4 carbon atoms in the alkyl radical, such as, for example, methyl and butyl glucoside, sugar alcohols having 5 to 12 carbon atoms, such as, for example, sorbitol or mannitol, sugars having 5 to 12 carbon atoms, such as, for example, glucose or sucrose, amino sugars, such as, for example, glucamine.

Solids which can be used are, for example, iron oxide pigments, titanium dioxide or zinc oxide particles and those additionally specified under “UV protectants”. Furthermore, it is also possible to use particles which lead to special sensory effects, such as, for example, nylon-12, boron nitride, polymer particles such as, for example, polyacrylate or polymethyl acrylate particles or silicone elastomers. Fillers which can be used include starch and starch derivatives, such as tapioca starch, distarch phosphate, aluminium starch or sodium starch, octenyl succinate, and pigments which have neither primarily a UV filter effect nor a colouring effect, for example Aerosils® (CAS No. 7631-86-9).

Examples of film formers which can be used, for example, for improving the water resistance are: polyurethanes, dimethicones, copolyol, polyacrylates or PVP/VA copolymer (PVP=polyvinylpyrrolidone, VA=vinyl acetate). Fat-soluble film formers which can be used are: e.g. polymers based on polyvinylpyrrolidone (PVP), copolymers of polyvinylpyrrolidone, PVP/hexadecene copolymer or the PVP/eicosene copolymer.

Pearlescence additives which can be used are, for example, glycol distearates or PEG-3 distearate.

Suitable deodorant active ingredients are, for example, odour concealers such as the customary perfume constituents, odour absorbers, for example the sheet silicates described in the patent laid-open specification DE 40 09 347, of these, in particular montmorillonite, kaolinite, illite, beidelite, nontronite, saponite, hectorite, bentonite, smectite, or also, for example, zinc salts of ricinoleic acid. Antimicrobial agents are likewise suitable for being incorporated. Antimicrobial substances are, for example, 2,4,4′-trichloro-2′-hydroxydiphenyl ether (Irgasan), 1,6-di-(4-chlorophenylbiguanido)hexane (chlorhexidine), 3,4,4′-trichlorocarbonilide, quaternary ammonium compounds, clove oil, mint oil, thyme oil, triethyl citrate, farnesol (3,7,11-trimethyl-2,6,10-dodecatrien-1-ol), ethylhexyl glyceryl ether, polyglyceryl-3 caprylate (TEGO® Cosmo P813, Evonik), and the effective agents described in the patent laid-open specifications DE 198 55 934, DE 37 40 186, DE 39 38 140, DE 42 04 321, DE 42 29 707, DE 42 29 737, DE 42 38 081, DE 43 09 372, DE 43 24 219 and EP 666 732.

Antiperspirant active ingredients which may be used are astringents, for example basic aluminium chlorides such as aluminium chlorohydrate (“ACH”) and aluminium zirconium glycine salts (“ZAG”).

Insect repellents which may be used are, for example, N,N-diethyl-m-toluamide, 1,2-pentanediol or Insect Repellent 3535.

Self-tanning agents which can be used are, for example, dihydroxyacetone and erythrulose.

Preservatives which can be used are, for example, mixtures of one or more alkyl paraben esters with phenoxyethanol. The alkyl paraben esters may be methyl paraben, ethyl paraben, propyl paraben and/or butyl paraben. Instead of phenoxyethanol, it is also possible to use other alcohols, such as, for example, benzyl alcohol or ethanol. Moreover, it is also possible to use other customary preservatives such as, for example, sorbic acid or benzoic acid, salicylic acid, 2-bromo-2-nitropropane-1,3-diol, chloroacetamide, diazolidinylurea, DMDM hydantoin, iodopropynyl butylcarbamate, sodium hydroxymethylglycinates, methylisothiazoline, chloromethylisothiazoline, ethylhexylglycerol or caprylyl glycol.

Conditioning agents which can be used are, for example, organic quaternary compounds, such as cetrimonium chloride, dicetyldimonium chloride, behentrimonium chloride, distearyldimonium chloride, behentrimonium methosulphate, distearoylethyldimonium chloride, palmitamidopropyltrimonium chloride, guar hydroxypropyltrimonium chloride, hydroxypropylguar, hydroxypropyltrimonium chloride, or quaternium-80 or else amine derivatives such as, for example, aminopropyldimethicones or stearamidopropyldimethylamines.

Perfumes which can be used are natural or synthetic odorants or mixtures thereof. Natural odorants are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots, (maize, angelica, celery, cardamon, costus, iris, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic odorant compounds are products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. It is possible to use mixtures of different odorants which together produce a pleasant scent note. Essential oils of low volatility, which are mostly used as aroma components, are also suitable as perfumes, e.g. sage oil, camomile oil, clove oil, Melissa oil, mint oil, cinnamon leaf oil, linden blossom oil, juniper berry oil, vetiver oil, olibanum oil, galbanum oil, labolanum oil and lavandin oil. It is also possible to use bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, fixolide NP, evernyl, iraldein gamma, phenylacetic acid, geranyl acetate, benzyl acetate, rose oxide, romillat, irotyl and floramat alone or in mixtures.

Dyes which can be used are the substances approved and suitable for cosmetic purposes, as are listed, for example, in the publication “Cosmetic Colourants” of the Dyes Commission of the German Research Society, Verlag Chemie, Weinheim, 1984, pp. 81 to 106. These dyes are usually used in concentrations of from 0.001 to 0.1% by weight, based on the total mixture.

Biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivatives), guanidine (and guanidine derivatives), pseudoceramides, essential oils, peptides, protein hydrolysates, plant extracts, bisabolol, allantoin, panthenol, phytantriol, idebenone, liquorice extract, glycyrrhizidine and idebenone, scleroglucan, β-glucan, santalbic acid and vitamin complexes. Examples of plant extracts are horsechestnut extract, camomile extract, rosemary extract, black and red currant extract, birch extract, rosehip extract, algae extract, green tea extract, aloe extract, ginseng extract, ginkgo extract, grapefruit extract, calendula extract, camphor, thyme extract, mangosteen extract, terminalia arjuna extract, oat extract, oregano extract, raspberry extract, strawberry extract, etc.

The biogenic active ingredients can also include the so-called barrier lipids, examples of which being ceramides, phytosphingosine and derivatives, sphingosine and derivatives, sphinganine and derivatives, pseudoceramides, phospholipids, lysophospholipids, cholesterol and derivatives, cholesteryl ester, free fatty acids, lanolin and derivatives, squalane, squalene and related substances.

Within the context of the invention, the biogenic active ingredients also include anti-acne, such as, for example, benzyl peroxide, phytosphingosine and derivatives, niacinamide hydroxybenzoate, nicotinaldehyde, retinol acid and derivatives, salicylic acid and derivatives, citronellic acid etc., and anti-cellulite, such as, for example, xanthine compounds such as caffeine, theophylline, theobromine and aminophylline, carnitine, carnosine, salicyloyl phytosphingosine, phytosphingosines, santalbic acid etc., as well as antidandruff agents such as, for example, salicylic acid and derivatives, zinc pyrithione, selenium sulphide, sulphur, cyclopiroxolamine, bifonazole, climbazole, octopirox and actirox etc., as well as astringents, such as, for example, alcohol, aluminium derivatives, gallic acid, pyridoxine salicylate, zinc salts, such as, for example, zinc sulphate, acetate, chloride, lactate, zirconium chlorohydrates etc. Bleaches such as kojic acid, arbutin, vitamin C and derivatives, hydroquinone, turmeric oil, creatinine, sphingolipids, niacinamide, etc. may likewise be included in the biogenic active ingredients.

Care additives which may be present are, for example, ethoxylated glycerol fatty acid esters, such as, for example, PEG-7 glycerol cocoate, or cationic polymers, such as, for example, polyquaternium-7 or polyglycerol esters.

Superfatting agents which can be used are substances such as, for example, lanolin and lecithin, and also polyethoxylated or acylated lanolin and lecithin derivatives, polyol fatty acid esters, monoglycerides and fatty acid alkanolamides, with the latter simultaneously serving as foam stabilizers.

Solvents which can be used are, for example, aliphatic alcohols such as ethanol, propanol or 1,3-propanediol, cyclic carbonates, such as ethylene carbonate, propylene carbonate, glycerol carbonate, esters of mono- or polycarboxylic acids such as ethyl acetate, ethyl lactate, dimethyl adipate and diethyl adipate, propylene glycol, dipropylene glycol, glycerol, glycerol carbonate or water.

Care formulations according to the invention can be used as skin care, face care, head care, body care, intimate care, foot care, hair care, nail care, dental care, lip care or mouth care product. Examples of hair care products are hair washing compositions, hair treatments, hair rinses, hair fluid, hair gel, hair tonic, hair wax, hair lacquer, hair spray, hair cream, hair mousse, hair balm, antidandruff shampoo. Examples of body care products are shower bath, cream bath, cream gel, shower oil, shower gel, washing gel, washing scrub, cleansing lotion, face mask, facial toner, facial scrub, eye cream, nightcream, cleansing mask, lotion pads, cleansing wipes, cleansing lotion, cleansing milk, cleansing gel, after shave gel, after shave balm, sun milk, after sun products, self-tanning products, foot lotion, foot spray, body lotion, body gel, body spray, body milk, body scrub, body oil, body butter. Examples of lip care products are lip balm, lip cream, lip care stick.

Care formulations according to the invention can be used in the form of an emulsion such as oil-in-water (O/W), water-in-oil (W/O) or water-in-silicone (W/S) emulsions, multiple emulsions such as W/O/W and O/W/O emulsions, also referred to as hydrodispersions or lipodispersions, a suspension, a solution, a cream, an ointment, a paste, a gel, an aerosol, a spray, a cleansing product, a make-up or sunscreen preparation or a facial toner or a stick, e.g. grease stick or water-containing stick.

Care formulations corresponding to the present invention have a conditioning effect on skin and hair. The invention thus provides the use of the care formulations for the conditioning of skin and/or hair.

The skin, by far the largest organ, serves as a barrier between the individual organism and the environment. This metabolically highly active protective layer is constantly subjected to oxidative stress which is caused by internal and external factors. These factors, in particular solar rays, produce harmful radicals, mostly reactive oxygen species (ROS). Each skin layer—from the outside inwards: stratum corneum, epidermis, dermis and hypodermis—has its own protective system against the attack of free radicals and has a different composition depending on the function of this layer. During oxidative stress, more free radicals and oxidants (ROS) are formed in the skin than the antioxidative protective systems can capture. The surplus ROS change the redox equilibrium of the skin cells. This activates redox-sensitive signal pathways which trigger a change in gene expression. Oxidative stress arises through an excess of ROS, for example as a result of UV exposure or ozone exposure. However, there are also cases of deficient endogenous free radical defence, for example in the event of a deficiency of antioxidative vitamins or enzyme defects in the antioxidative defence. This form of oxidative stress can be intensified by pathophysiological inflammatory reactions, and the metabolism of the cell. The long-wave UVA radiation can penetrate as far as into deep skin layers. It can thus react not only with the epidermal cells, but also with the fibroblasts of the dermis and cause the formation of free radicals therein. The short-wave UVB radiation, on the other hand, is for the large part already absorbed in the epidermis and changes primarily DNA and proteins in epidermal keratinocytes and Langerhans cells. To a lesser degree, UVB radiation also stimulates the formation of free radicals through hydrolysis. Thus, lipids, proteins and nucleic acids are damaged by different UV wavelengths in different layers.

If human hair is to be coloured permanently, only oxidizing hair colouring processes are suitable in practice. During oxidative hair colouring, the formation of the dye chromophore takes place through reaction of precursors (phenols, aminophenols, or rarely also diamines) and bases (in most cases p-phenyldiamine) with the oxidizing agent, in most cases hydrogen peroxide; hydrogen peroxide concentrations around 6% are usually used here. It is usually assumed that, besides the colouring effect, a bleaching effect due to the hydrogen peroxide also takes place. In oxidatively coloured human hair it is possible to detect microscopic holes in the positions where melanine granules were present, similarly to in the case of bleached hair. Oxidizing agents such as hydrogen peroxide reacts not only with the dye precursors, but also with the hair substance and may under certain circumstances result in damage to the hair.

These damaging processes of the skin and hair described above can be counteracted by the care formulations according to the invention.

The invention therefore further provides the use of the care formulations according to the invention for the treatment and/or prophylaxis of skin ageing and/or hair damage caused by oxidative stress.

The invention therefore likewise provides the use of the care formulations according to the invention for reducing hair and/or skin damage caused by environmental toxins or UV-induced hair and/or skin damage.

Stretch marks are visible phenomena in the subcutis which are formed as the result of severe stretching of the tissue—Striae cutis atrophicae or Striae cutis distensae (from Latin Striae=marks, cutis=skin, atrophicae=atrophic, distensae=overstretched). In the course of a pregnancy, the appearance of stretch marks is physiological; they are referred to as pregnancy stretch marks (Latin Striae gravidarum). Moreover, stretch marks may be symptoms of disorders, such as obesity and can occur during Cushings syndrome or arise as a side-effect of medicaments. The coloration is caused by transparent blood vessels. The marks preferably occur in tissues where larger amounts of fat are stored, such as abdomen, hips, buttocks, upper arms and breasts. Predisposing factors are a connective tissue weakness and considerable weight gain. Additionally, the elasticity of the skin is reduced during a pregnancy as a result of hormonal influences. The connective tissue which is responsible for the elasticity of the skin consists of a network of collagenous fibres. Overstretching of the connective tissue leads to irreparable tears in the subcutis, which lead to externally visible blue-reddish marks. Over the course of time, the marks fade, but continue to remain visible as pale scars.

The horny layer (Stratum corneum) is a thin (ca. 10 μm), tear-resistant and almost completely impermeable film which covers the epidermis like a plastic membrane. In its entirety, it is carrier of the barrier function. The horny layer is resistant to physical and chemical noxae (acids, less so alkali), but is relatively sensitive towards organic solvents and detergents. Due to the protein character, the horny layer is hygroscopic (water-attracting). Relatively prolonged exposure to water results in swelling and thus to a drastic change in the physical properties, such as, for example, decrease in the tear strength and impairment of the impermeability. The barrier function of the horny layer is more incomplete. Minimum material exchange between organism and environment (e.g. Perspiratio insensiblis) is afforded. On the other hand, any low molecular weight substance can penetrate to a low degree into the skin, lipid-soluble substances being preferred over water-soluble substances. Astringent properties of substances tighten the tissue and the vascular permeability is reduced, penetration by germs is prevented and the resistance of the skin increases. They enter into bonds with the keratin of the horny cells and crosslink these. As a result, a pulling together of the uppermost horny skin layer is achieved. The skin becomes mechanically firmer and the barrier effect increases. This leads to the reduction in inflammatory skin reactions. Since the care formulations according to the invention have astringent properties, the invention further provides the use of the care formulations according to the invention for tightening and/or firming the skin.

The invention therefore likewise provides the use of the care formulations according to the invention for increasing the resistance of the skin.

Care formulations according to the invention generally have a skin-calming and anti-inflammatory action, the invention therefore further provides a use of the care formulations according to the invention for the nontherapeutic treatment and/or prophylaxis of inflammatory reactions on and/or in the skin.

Since the care formulations according to the invention lead to an improvement in the ability of the skin to retain moisture, the invention further provides the use of the care formulations according to the invention for increasing the moisture content of the skin, for reducing and smoothing skin wrinkles, for colouring the skin evenly or for producing a uniform appearance of the skin surface.

Since the care formulations according to the invention lead to improved tensile strength and/or substantivity and/or elasticity of the hair, the invention further provides the use of the care formulations according to the invention for increasing the tensile strength and/or substantivity and/or elasticity of the hair.

The present invention is described by way of example in the examples listed below, without any intention to limit the invention, the scope of application of which arises from the entire description and the claims, to the embodiments specified in the examples.

Unless stated otherwise, all of the stated percentages (%) are percentages by mass.

The figures below are part of the examples:

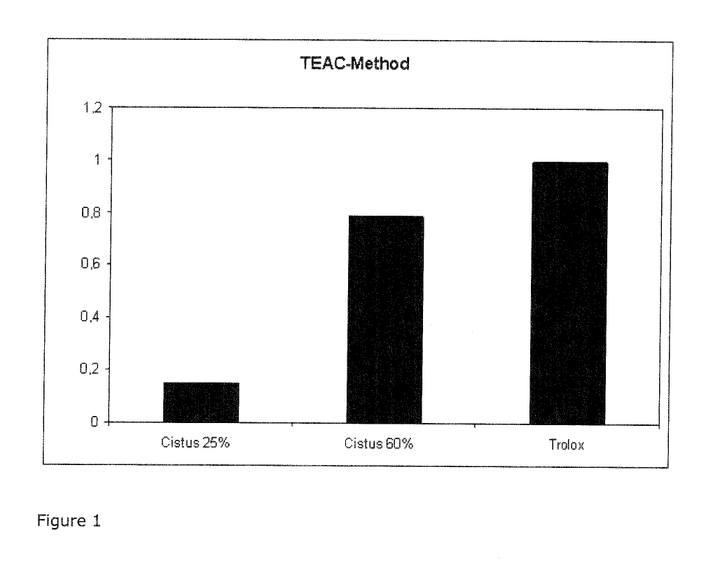

FIG. 1: Results of the Teac test

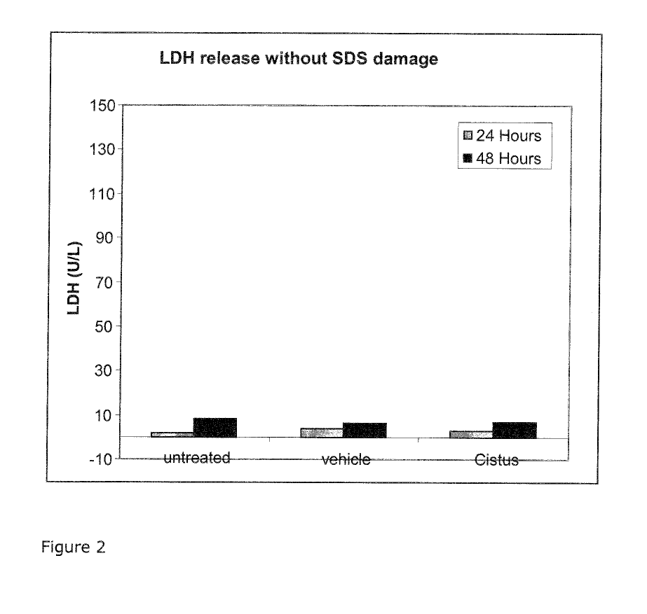

FIG. 2: Results of LDH release without SDS

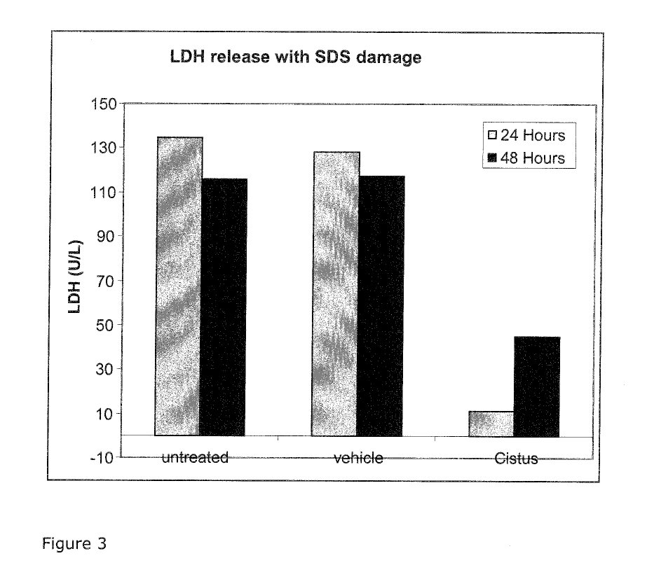

FIG. 3: Results of LDH release with SDS

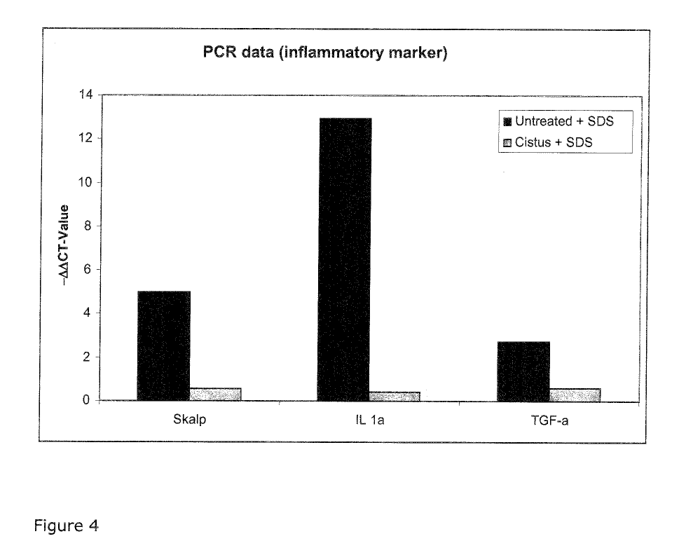

FIG. 4: Results of RT-PCR inflammatory marker

### EXAMPLES

### Example 1

### Preparation of the Extract, According to the Invention

15.5 kg of starting drug Herba Cistus incanus L ssp. tauricus with a starting content of 14.6 catechin equivalent percent are exhaustively extracted in the percolator at 40° C. twice with ethanol 40% (V/V) to 1:8. The eluates separated off from the drug are combined, filtered and freed from the solvent gently in vacuo at 50° C. 6 kg of aqueous spissum extract with a dry substance fraction of 65% (=3.9 kg of native extract fraction) are obtained. The polyphenol content is 31.4 catechin equivalent percent calculated on the native extract.

5.8 kg of the spissum extract (3.77 kg of native extract fraction) are back-dissolved with demineralized water to 20% dry substance fraction and intensively homogenized with stirring for 60 minutes. The mixture is then left to stand for 4 h at 10-15° C. A sedimented, insoluble bottom phase results (5% of the total amount). This residue (tannin fraction) revealed a polyphenol content of 22% based on the native extract.

The supernatant (product phase) is drawn off from above and filtered over a CP1KS filter plate until clear (quantitative yield 95%). The polyphenol content was 32.0 catechin equivalent percent based on the native extract. The extract solution comprising 3.58 kg of native extract as ca. 20% strength solution is introduced onto a column filled with 50 l of adsorber resin (Amberlite® XAD7HP). The pass is separated off and discarded. The column is then rinsed clean with 3 bed volumes (150 l) of water. The elution of the adsorbed secondary cistus ingredients takes place with 100 l of ethanol 96% (V/V). The ethanolic-aqueous eluate is collected, filtered until clear and freed from solvent via vacuum concentration and simultaneously steamed to give the spissum. 2 kg of spissum with a dry substance fraction of 65% (=1.3 kg of native extract) are obtained. The polyphenol content was 65.0% catechin equivalent percent based on the native extract. The monomers fraction (theogallin, epigallocatechin, catechin, epicatechin and epigallocatechin gallate) is 3.6%.

### Example 2

### Teac Test

The extract from Example 1 was used in the following; it has a polyphenol content of 60% catechin equivalent percent, determined as described above, and is called “Cistus 60%” below.

The antioxidative potential of Cistus 60% was determined using a Teac test, carried out in accordance with Buenger et al.; International Journal of Cosmetic Science, 2006, 28, 135-146.

For comparison with the prior art, a conventional aqueous Cistus extract with 25% polyphenol content catechin equivalent percent (“Cistus 25%”) was used. This is sold by extract manufacturer Finzelberg under Art. 0 431 100.

FIG. 1 shows that Cistus 60% has a more than four times higher antioxidative capacity than the comparison extract although the content of polyphenols is only 2.4 times higher.

### Example 3

### Antioxidative Potential in Accordance with the ORAC Method

ORAC (Oxygen Radical Absorption capacity) is an international standardized method for determining antioxidative potential. Using this method, a differentiation into hydrophilic and lipophilic fractions of the antioxidative effect is possible. For better comparability, the total capacity is also quoted in the unit of the Trolox equivalent (TE). The procedure is described at www.orac-europe.com.

Cistus extract (60% catechin equivalent percent)=5.1 μmol of TE/mg

Cistus extract (25% catechin equivalent percent=1.9 μmol of TE/mg

The Cistus extract with 60% catechin equivalent percent enriched according to the invention has an extraordinarily high ORAC potential which is higher than the prior art by a factor of 2.7.

### Example 4

### LDH Release, Inflammatory Markers in Skin Models

To characterize regenerative properties, in the study, skin and epidermis models from SkinEthic RHE, Nice, France were topically treated with Cistus 60% for one hour. The tissue models were then subjected to defined damage with SDS (30 μl, 0.25%, 40 minutes) and Cistus 60% was applied for a further 48 hours. By reference to LDH measurements, and RT-PCR measurements, the aim was to establish whether a protection could be imparted to the skin models and whether an anti-inflammatory effect is present.

### Treatment of the Skin Models

The in vitro reconstructed human epidermis model (Reconstructed Human Epidermis RHE/S/17, Lot 07022A 0809, Skinethic Nizza, France) were, after receipt, removed from the freight packaging and transferred to 6-well cell culture plates with in each case 300 μl of maintenance medium. After preincubation for four hours at 37° C. and 5% CO2, the cell culture medium was exchanged for 1000 μl of fresh maintenance medium and a further adaption phase was carried out overnight at 37° C. and 5% CO2. On the day of testing, 100 μl of 0.2% aqueous Cistus 60% solution and/or vehicle (water) (both filtered through 0.45 μm spray filter) were applied in triplicate to the dry Stratum corneum of the 3D epidermis models and incubated at 37° C. and 5% CO₂ over a period of 1 hour. The excess test substance was then removed from the surface of the reconstructed epidermises. Damage of the skin models takes place with 30 μl, 0.25% strength SDS for 40 minutes. Finally, the skin models are washed 20 times with PBS buffer. For all skin models, a media exchange (1000 μl maintenance medium) was then carried out.

Renewed application of 100 μl of 0.2% aqueous Cistus 60% solution for 48 hours. After 24 hours medium exchange and removal of 1 ml of medium for the LDH determination. After a further 24 hours, removal again of 1 ml of medium for the LDH determination. The skin models were placed in 500 μl of RNA later.

Medium exchange was carried out: 2.5 h after introducing the cells into the medium, before applying the test substance, following damage by SDS and after 24 h for testing viability. Furthermore, the LDH release into the medium was determined before applying the test substance, after 24 h and after 48 h:

### Lactate Dehydrogenase Release (LDH Release)

The appearance of LDH in the cell culture medium is a sure sign of damage to the cytoplasmatic membrane of the cells and thus damage to the epidermal cell layer. Furthermore, it is known that an emergence of this enzyme represents the “point of no return” for the cell, and thus indicates an irreversibility of the damage.

### Determination of the LDH Release

The quantification of the LDH activity was carried out using a commercially available test kit and took place in accordance with manufacturer's instructions. When determining the activity of the LDH*, an LDH dilution series was also entrained as standard. (*LDH test kit and standard, Roche Diagnostics, Mannheim, Germany).

The results are shown in FIGS. 2 and 3 and show that Cistus 60% significantly reduces LDH release upon damage in skin models.

As anti-inflammatory marker, the expression of the genes in SKALP-F, IL-1α and TNF-α was determined by means of RT-PCR as follows:

Isolation of RNA from the Skin Models

The isolation of the entire RNA from the skin models is carried out using the RNeasy mini kit. For the analysis, the frozen skin models are heated to room temperature and treated with 1 ml of Qiazol solution. The skin model is homogenized in the tissue lyser for 5 min at 16,000 Hertz using a 5 mm steel ball. The solution is left to stand for 5 min at room temperature and then admixed with 200 μl of chloroform. This solution is mixed for 15 s and again left to stand at room temperature for 3 min. The suspension is centrifuged for 15 min at 4° C. and 12,000 rpm and the upper phase is placed in a new Eppendorf cap. The removed amount is admixed with the same removed volume (ca. 600 μl) of ethanol (70%). 700 μl of liquid are removed, transferred to an RNeasy mini column and centrifuged for 15 s at 13000 rpm, the liquid can be discarded. With the remainder of the solution, the procedure is the same. 700 μl of RW1 buffer are then added and the mixture is centrifuged for 15 s at 13,000 rpm, the liquid can be discarded. The columns are placed on to a new tube (2.2 ml) and treated with 500 μl of RPE buffer and centrifuged again for 15 s at 13000 rpm, the liquid is discarded. 500 μl of RPE buffer are then added, the mixture is centrifuged for 2 min at 13,000 rpm, the transfer columns are positioned on 1.5 ml Eppendorf caps (2 ml) and 50 μl of RNase-free water are added, then centrifugation is carried out for one minute at 13,000 rpm. For the measurement, 10 μl of RNA solution are admixed with 90 μl of water and the absorption is measured at a wavelength of 260 nm. An absorption (A260) of 1.00 corresponds to a concentration of 50 μg/ml of double-stranded DNA, 33 μg/ml of short, single-stranded DNA or 40 μg/ml of RNA. The remaining 40 μl of solution are divided (in each case 10 μl) and stored in the freezer at −80° C.

### First Strand Synthesis

Using the first-strand-cDNA synthesis kit for the RT-PCR, reverse transcription is carried out.

In each case, 100 ng of RNA from the samples are admixed with 1 μl of random hexamer (50 μM) and 1 μl of DEPC water and brought to a volume of 10 μl with water. This solution is incubated at 65° C. for five minutes and then stored on ice for one minute. To test for systematic error sources, water is used as blank sample in all investigations. 10 μl of cDNA synthesis mix are then added to each solution, which is mixed and heat-treated for 10 minutes at 25° C., for 50 minutes at 50° C. and for 5 minutes at 85° C. and then stored on ice. 1 μl of RNase H is added to each solution, which is incubated for 20 minutes at 37° C. The solution can then be frozen at −80° C. or be used for a PCR measurement.

Preparation of the cDNA synthesis mix (storage at −20° C.) for the first strand synthesis:

10* RT buffer [2 μl]

### 25 mM MgCl2 [4 μl]

### 0.1 M DTT [2 μl]

### RNaseOUT (40 U/μl) [1 μl]

### SuperScript III RT (200 U/μl) [1 μl]

### PCR

1.5 μl of template (cDNA) are removed and the following solutions are added:

template composition for the PCR, primer

### 2* SYBR Green MasterMix 25 [μl]

Primer-F (100 pmol/μl) 0.15 [μl]
Primer-R (100 pmol/μl) 0.15 [μl]
RNase free water 23.2 [μl]
Initial denaturing: 15 minutes at 95° C.

### Amplification:

### Denaturing: 15 s at 94° C.

### Annealing: 30 s at 50° C.

### Extension: 30 s at 72° C.

For a number of 44 cycles with subsequent melting point determination, the duration of the complete PCR was 2.5 hours. The data was evaluated using the Opticon Monitor Analysis Software, Version 1.05 from MJ Research.

Oligonucleotides Used from MWG (Forward Primer)

| Forward primer sequence (5′-3′) | |
|---|---|
| GAPDH | 5′-GAAGGTGAAGGTCGGAGTCAACG-3′ |
| SKALP | 5′-CATGAGGGCCAGCAGCTT-3′ |
| IL-1α | 5′-GTCTCTGAATCAGAAATCCTTCTA-3′ |
| TNF-α | 5′-CTCTGGCCCAGGCAGTCAGA-3′ |

Oligonucleotides Used from MWG (Reverse Primer)

| Reverse primer sequence (5′-3′) | |
|---|---|
| GAPDH | 5′-AGTCCTTCCACGATACCAAAGTTG-3′ |
| SKALP-F | 5′-TTTAACAGGAACTCCCGTGACA-3′ |
| IL-1α | 5′-CATGTCAAATTTCACTGCTTCATC-3′ |
| TNF-α | 5′-GGCGTTTGGGAAGGTTGGAT-3′ |

The results are shown in FIG. 4 and show that Cistus 60% leads to a significant reduction of inflammatory markers upon damage in skin models.

### Example 5

### Example Formulations

Example formulations are described below; stated percentages are percentages by mass and are based on the total mass of the example formulation. To prepare the formulations, customary formulation processes known to the person skilled in the art were used. “Cistus” stands in each case by way of representation for the extract obtained in Example 1.

O/W formulation

| | |
|---|---|
| Phase A | |
| Polyglycerol-3 Methylglucose Distearate | 3.0% |
| Glyceryl Stearate | 2.0% |
| Stearyl Alcohol | 1.0% |
| Decyl Cocoate | 10.0% |
| Cetearyl Ethylhexanoate | 9.0% |
| Phase B | |
| Glycerol | 3.0% |
| Cistus | 0.2% |
| Water | ad 100% |
| NaOH (10%) | pH 5.5-6.0 |

W/O Formulation

| | |
|---|---|
| Phase A | |
| Cetyl PEG/PPG-10/1 Dimethicone | 2.0% |
| Polyglyceryl-4 Isostearate | 1.0% |
| Beeswax | 1.2% |
| Hydrogenated Castor Oil | 0.8% |
| Diethylhexyl Carbonate | 9.5% |
| Caprylic/Capric Triglyceride | 6.0% |
| Cetearyl Ethylhexanoate | 4.0% |
| Phase B | |
| Sodium Chloride | 0.5% |
| Propylene Glycol | 2.0% |
| Cistus | 0.1% |
| Water | ad 100% |
| NaOH (10%) | pH 5.5-6.0 |

Massage oil

| | |
|---|---|
| Stearyl alcohol | 2.0% |
| Petrolatum | 4.0% |
| Dimethicone | 2.0% |
| Isopropyl palmitate | 6.0% |
| Cetylstearyl alcohol | 4.0% |
| PEG-40 hydrogenated castor oil | 2.0% |
| Cistus | 0.2% |
| Glycerol | 3.0% |
| Water | ad 100 |

Shower gel

| | |
|---|---|
| PEG-7 Glyceryl Cocoate | 2.0% |
| PEG-40 Hydrogenated Castor Oil | 2.5% |
| Sucrose Cocoate | 2.5% |
| Perfume | 0.5% |
| Water | ad 100% |
| Cistus | 0.2% |
| Cocamidopropyl Betaine | 10.5% |
| Sodium Lactate, Sodium PCA Glycine, Fructose, Urea, | 1.0% |
| Niacinamide, Inositol, Sodium benzoate, Lactic Acid, | |
| Glycol Distearate, Steateth-4 | 2.0% |

Shampoo

| | |
|---|---|
| Sodium Laureth Sulphate (28%) | 35% |
| Perfume | 0.5% |
| Water | ad 100% |
| Cistus | 0.2% |
| Quaternium-80 | 0.5% |
| PEG/PPG-4/12 Dimethicone | 0.5% |
| Cocoamidpropyl Betaine | 11.0% |
| PEG-120 Methyl Glucose Dioleate | 0.9% |

1-12. (canceled)

13. A method of preparing an extract from cistus comprising:
extracting a plant extract from Herba Cistus ssp. L. with an extractant selected from the group consisting of water, alcohols and mixtures thereof;
removing extraction residues from said plant extract;
at least partially removing the extractant from said plant extract;
redissolving said plant extract in an aqueous solvent and removing insoluble constituents therefrom; and
subjecting said redissolved plant extract to solid-phase extraction to provide an enriched plant extract.

14. The method of claim 13 further comprising adding said enriched plant extract as an active ingredient to a care formulation.

15. The method of claim 13 wherein said care formulation is topical.

16. The method of claim 14 wherein said care formulation is a cosmetic formulation, a dermatological formulation or a pharmaceutical formulation.

17. The method of claim 14 wherein said enriched plant extract comprises from 0.01 mass percent to 20 mass percent of care active ingredient, based on the total mass of the care formulation.

18. A method of treating human or animal body parts comprising:
preparing an extract from cistis;
adding said extract to a care formulation; and
applying said care formulation to skin, hair or both skin and hair, wherein said extract is prepared by:
extracting a plant extract from Herba Cistus ssp. L. with an extractant selected from the group consisting of water, alcohols and mixtures thereof;
removing extraction residues from said plant extract;
at least partially removing the extractant from said plant extract;
redissolving said plant extract in an aqueous solvent and removing insoluble constituents therefrom; and
subjecting said redissolved plant extract to solid-phase extraction to provide an enriched plant extract.

19. The method of claim 18 wherein said skin, hair or both skin and hair have a disorder caused by oxidative stress, environmental toxins or UV exposure.

20. The method of claim 18 wherein said applying said care formulation is to skin, and said skin has an increase in at least one of tightness, firmness and resistance after said applying.

21. The method of claim 18 wherein said applying said care formulation is to skin having an inflammatory reaction disorder.

22. The method of claim 18 wherein said applying said care formulation is to hair, and said hair has an increased in at least one of tensile strength, substantivity and elasticity after said applying.
